(19) United States
(12) Patent Application Publication
(10) Pub. No.: US 2024/0371490 A1
(43) Pub. Date: Nov. 7, 2024

(54) ADVANCED MODELING FOR BIOLOGICS

(71) Applicant: Children's Hospital Medical Center, Cincinnati, OH (US)

(72) Inventor: Phillip Minar, Montgomery, OH (US)

(21) Appl. No.: 18/654,650

(22) Filed: May 3, 2024

Related U.S. Application Data
(60) Provisional application No. 63/464,371, filed on May 5, 2023.

(51) Int. Cl. G16H 20/17; A61K 39/00; A61P 1/00; A61P 29/00; C07K 16/24

(52) U.S. Cl. CPC G16H 20/17; A61P 1/00; A61P 29/00; C07K 16/241; A61K 2039/505

(57) ABSTRACT

Disclosed are methods for determining an initial or subsequent dose of an anti-tumor necrosis factor α (anti-TNFα) biologic for administering to an individual having an inflammatory condition, based on, in part, determining in the individual a level of one or more time-varying covariates selected from weight, albumin, erythrocyte sedimentation rate (ESR), neutrophil CD64 (nCD64) expression, and combinations thereof, and applying a correction factor to account for the changes in covariates over time. The inflammatory condition may be one selected from an IBD, such as Crohn's Disease (CD) or Ulcerative Colitis (UC), or inflammatory conditions such as uveitis or rheumatoid arthritis.

### CROSS-REFERENCE TO RELATED APPLICATIONS

This application is a U.S. non-provisional application which claims priority to and benefit of U.S. Provisional Ser. No. 63/464,371, filed May 5, 2023, the contents of which are incorporated in their entirety for all purposes.

### STATEMENT REGARDING FEDERALLY-SPONSORED RESEARCH

This invention was made with government support under DK132408 awarded by the National Institutes of Health. The government has certain rights in the invention.

### BACKGROUND

Model-informed precision dosing (MIPD) is a strategy in pharmacotherapy that utilizes mathematical models to optimize drug dosing regimens for individual patients. These models may incorporate various factors such as pharmacokinetics, pharmacodynamics, patient demographics, genetics, and other relevant variables to tailor the dosing regimen to achieve desired therapeutic outcomes. Ideally, MIPD may be used to provide individualized dosing regimens while reducing adverse effects, optimizing drug efficacy, and improving cost effectiveness.

Inflammatory bowel disease (IBD), including Crohn's disease (CD) and ulcerative colitis (UC), is a chronic inflammatory condition of the gastrointestinal tract with approximately one quarter of patients diagnosed before age 20. Both the incidence and prevalence of pediatric-onset IBD has been increasing internationally in the twenty-first century. Pediatric-onset of CD typically exhibits a more aggressive phenotype compared to adult-onset CD leading to a higher rate of intestinal complications. Currently anti-TNFα biologics (infliximab and adalimumab) are the only biologics approved by the United States Food and Drug Administration (FDA) and the European Medicines Agency (EMA) for the treatment of children (6-17 years old) with moderate to severe CD or UC. Despite the recent FDA/EMA approval of more novel advanced therapies for adults with IBD, the anti-TNFα biologics remain first-line therapy for management of moderate to severe pediatric CD in North America⁴ and are second-line for moderate to severe UC.

Routine use of proactive therapeutic drug monitoring (TDM) for children receiving anti-TNFα biologics has been used to achieve superior rates of clinical and biochemical remission, a decrease in the incidence of high-titer antidrug antibodies (ADA) and improved drug durability. Not only has TDM been a key advancement, but optimized induction dose selection and the attainment of early (week6, dose3) and postinduction infliximab trough concentrations has been associated with improved early clinical and biochemical outcomes. Model informed precision dosing to personalize biologics, such as infliximab, has been shown to improve dose optimization strategies and clinical outcomes during the induction and maintenance phases of treatment. However, there is room in the art for improvement of MIPD methods and methods for treating one or more of the aforementioned disease states.

### BRIEF SUMMARY

Disclosed are methods for determining a dose of an anti-tumor necrosis factor α (anti-TNFα) biologic for administering to an individual having an inflammatory condition, based on, in part, determining in the individual a level of one or more time-varying covariates selected from weight, albumin, erythrocyte sedimentation rate (ESR), neutrophil CD64 (nCD64) expression, and combinations thereof. An initial dose, or a subsequent dose, for example one or more doses administered to an individual during an induction phase, during a maintenance phase, or both, may be determined using the disclosed methods. The inflammatory condition may be an IBD, such as Crohn's Disease (CD) or Ulcerative Colitis (UC), or may be an inflammatory condition such as uveitis or rheumatoid arthritis.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only and not intended to limit the scope of the disclosure in any way.

FIGS. 1A-1D. Estimate of time-varying changes in the four covariates of infliximab clearance. The Emax method was used to evaluate the percent change in serum albumin (ALB) (1A), weight (1B), the erythrocyte sedimentation rate (ESR) (1C), and the neutrophil CD64 ratio (nCD64) (1D) over the first four doses (dose1, dose2, dose3 and dose4).

FIGS. 2A-2B. Simulated infliximab pharmacokinetic profiles. The predicted concentration-time curves were simulated for a 47 kg patient receiving 5 mg/kg (2A) and 10 mg/kg (2B). The dashed line (- - -) is the predicted concentration curve using the baseline covariates alone. The solid line (---) is the predicted concentration curve using the time-varying covariates with the Emax model.

FIG. 3. Observed and predicted infliximab trough concentrations using different modeling approaches. At each dose (2, 3 and 4), the median (solid line) and the observed range of infliximab concentrations were compared to the median (range) predicted infliximab concentrations using the four baseline (pre-treatment) covariates only or the time-varying covariates estimated with the Emax model. For each dose, the left data set is the observed concentration, the middle data set is predicted concentrations, baseline covariates only, and the right data set is predicted concentrations using the Emax time-varying covariate model. NS, not significant; **, p<0.01; ***, p<0.001.

FIGS. 4A-4C. Comparison of observed and predicted infliximab trough concentrations using the different modeling approaches and the overall predictive performance of each approach. For each dose, a box and whiskers plot was made for observed (red) and predicted infliximab concentrations (blue). Additionally, bias (%) and precision (%) was calculated at (a) dose2, (b) dose3 and (c) dose4 using the listed scenarios. Bias (%), a measure of accuracy, and precision (%) were calculated and comparisons between the different modeling approaches. NS, not significant; *, p<0.05; **, p<0.01; ***, p<0.001.

FIGS. 5A-5B. Simulated infliximab pharmacokinetic profiles using baseline covariates alone and the time-varying covariates for a 47 kg patient receiving (A) 5 mg/kg and (B) 10 kg/kg of infliximab.

FIGS. 6A-6B. Simulated infliximab pharmacokinetic profiles using baseline covariates alone and the patient's weight as the only time-varying covariate for a 47 kg patient receiving (A) 5 mg/kg and (B) 10 mg/kg of infliximab. Baseline covariates are indicated via the dashed line, while weight only is indicated using a solid line.

FIG. 7 is an exemplary display showing example covariates for a given biologic, in this case infliximab, which may be used for carrying out the disclosed methods.

FIG. 8 is an exemplary display showing the output for the model using the model correction.

FIG. 9 is an exemplary display showing the output for the model without the model correction.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in predicting a targeted dose of infliximab for administration to an individual in need thereof.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives.

“Treat” or “treating” means to administer a therapeutic agent, such as a therapeutic described herein, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. Those of ordinary skill in the art will be aware of a variety of routes that may, in appropriate circumstances, be utilized for administration to a subject, for example a a human subject. For example, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children, for example pediatric patients under the age of 18 years of age, or pre-pubescent pediatric patients.

Disclosed herein are methods and systems for improved dosing of an individual in need of a biologic-based therapy, in particular in an individual having an inflammatory bowel disease.

In aspects, disclosed is a method for determining a dose of an anti-TNFα biologic for administering to an individual having an inflammatory disorder. The dose, ideally, provides an effective amount of the anti-TNFα biologic for providing a therapeutic and/or prophylactic effect. In aspects, the dose that is determined is an initial dose of an anti-TNFα biologic that provides, or is intended to provide, an effective amount of the anti-TNFα biologic for providing a therapeutic and/or prophylactic effect. In aspects, the dose that is determined is a subsequent dose that follows the initial dose of the anti-TNFα biologic, that provides, or is intended to provide, an effective amount of the anti-TNFα biologic for providing a therapeutic and/or prophylactic effect. In aspects, the dose that is determined is any dose of an anti-TNFα biologic administered during the induction period that provides, or is intended to provide, an effective amount of the anti-TNFα biologic for providing a therapeutic and/or prophylactic effect. In aspects, the dose that is determined is any dose of an anti-TNFα biologic administered during the maintenance period that provides, or is intended to provide, an effective amount of the anti-TNFα biologic for providing a therapeutic and/or prophylactic effect. The dose identified by the disclosed methods may be provided as part of a personalized treatment plan which minimizes the likelihood of under-dosing or over-dosing the individual, thus maximizing the likelihood of an effective treatment while avoiding waste and/or negative consequences such as inflammation that may result from over-dosing of a therapeutic.

In one aspect, the individual is diagnosed with or suspected of having an inflammatory disorder that is an inflammatory bowel disease (IBD). The IBD may be selected from moderate CD, severe CD, moderate UC, severe UC, and combinations thereof. In other aspects, the individual may be one who is suspected of having an IBD selected from moderate CD, severe CD, moderate UC, severe UC, and combinations thereof.

In one aspect, the individual is diagnosed with or suspected of having an inflammatory disorder that is rheumatoid arthritis.

In one aspect, the individual is diagnosed with or suspected of having an inflammatory disorder that is uveitis.

In one aspect, the method may comprise:
a) determining, in the individual, a level of one or more time-varying covariates;
b) predicting a future concentration of the anti-TNFα biologic based on the one or more time-varying covariate levels determined in step (a);
c) determining, based on the predicted future concentration of step (b), a dose of the anti-TNFα biologic based on the predicted future concentration obtained in (b).

The dose is, in general, a dose that provides, or is intended to provide, an effective dose of the anti-TNFα biologic to the individual in need thereof. In aspects, the dose is an initial dose. In aspects, the dose is a subsequent dose (administered following the initial dose). In aspects the dose is any dose administered during the induction period. As used herein, the term “induction phase” refers to the initial dosing of an active agent over a period of time, during which a therapeutic agent is administered at a concentration and interval designed to achieve a rapid therapeutic effect. The induction phase typically involves higher doses or doses administered at an increased interval as compared to the maintenance phase. The induction phase may be carried out, for example, over a period of from about 1 day to about 10 weeks, or about 1 week to about 9 weeks or about 2 weeks to about 8 weeks, or about 3 weeks to about 7 weeks. For example, an induction phase may be carried out for about two weeks, or about three weeks, or about four weeks, or about five weeks, or about six weeks, or about seven weeks, or about eight weeks, or about nine weeks, or ten weeks or more. In general, following the induction phase, the therapeutic is administered at a reduced interval but which maintains the therapeutic effect (the maintenance phase).

In one aspect, the time-varying covariate may be selected from weight, albumin, erythrocyte sedimentation rate (ESR), neutrophil CD64 (nCD64) expression, and combinations thereof. In one aspect, the one or more time-varying covariates may comprise at least two of weight, albumin, erythrocyte sedimentation rate (ESR), neutrophil CD64 (nCD64) expression. In one aspect, the one or more time-varying covariates may comprise at least three of weight, albumin, erythrocyte sedimentation rate (ESR), neutrophil CD64 (nCD64) expression. In one aspect, the one or more time-varying covariates may comprise each of weight, albumin, erythrocyte sedimentation rate (ESR), neutrophil CD64 (nCD64) expression. In aspects, the time-varying covariate may be selected from at least one of, or at least two of, or each of albumin, erythrocyte sedimentation rate (ESR), neutrophil CD64 (nCD64) expression, and combinations thereof.

In one aspect, the time-varying covariate that is measured is albumin. Human albumin is a small globular protein with a molecular weight of 66.5 kilodaltons (kDa). It consists of 585 amino acids which are organized into three repeated homologous domains and are made up of two separate sub-domains, A and B.An albumin level may be determined via methods known in the art. For example, serum albumin can be measured via standard serum laboratory testing as noted in Moman R N, Gupta N, Varacallo M. Physiology, Albumin. [Updated 2022 Dec. 26]. In: StatPearls [Internet]. Treasure Island (FL): StatPearls Publishing; 2024 January-. Available from: www.ncbi.nlm.nih.gov/books/NBK459198/.

In one aspect, the time-varying covariate that is measured is the erythrocyte sedimentation rate (sedimentation rate, sed rate, or ESR for short). An ESR level may be determined via methods known in the art. For example, the Westergren method, which measures the distance (in millimeters) at which red blood cells in anticoagulated whole blood fall to the bottom of a standardized, upright, elongated tube over one hour due to the influence of gravity may be used. The tube used for the test is called the Westergren tube, which is made of either glass or plastic, with an internal diameter of 2.5 mm and lengths of 190 to 300 mm. See, e.g., Tishkowski K, Gupta V. Erythrocyte Sedimentation Rate. [Updated 2023 Apr. 23]. In: StatPearls [Internet]. Treasure Island (FL): StatPearls Publishing; 2024 January-. Available from: www.ncbi.nlm.nih.gov/books/NBK557485/.

In one aspect, the time-varying covariate that is measured is a Neutrophil CD64 (nCD64) level. CD64 is a high-affinity IgG receptor, also known as FcγR1 widely expressed on monocytes and, to a lesser extent, resting neutrophils. Neutrophil CD64 (nCD64) is a remarkably accurate and selective marker for systemic infections and sepsis in all age groups. The nCD64 level may be determined via methods known in the art, for example as described in van de Ven et al.; COVPACH study group. Point-of-care neutrophil CD64 as a rule in diagnostic test for bacterial infections in the emergency department. BMC Emerg Med. 2023 Mar. 14; 23 (1): 28. doi: 10.1186/s12873-023-00800-2. PMID: 36915043; PMCID: PMC10010956.

The step of predicting a future concentration of the anti-TNFα biologic may include modeling the future level of an anti-TNFα biologic in the individual, based on both a time-varying covariate level and a correction factor applied to the population PK model for the therapeutic. In one aspect, the time-varying co-variate level is determined prior to treatment with an anti-TNFα biologic. In this aspect, the method is used to determine an initial dosage to be provided to the individual. In another aspect, the time-varying co-variate level is determined after an initial dose is administered, and the time-varying co-variate level is used to optimize (determine) a subsequent dose. The subsequent dose may be, for example, one or more of a second dose, a third dose, a fourth dose, a fifth dose, a sixth dose a seventh dose, an eighth dose, a ninth dose, a tenth dose, or a dose following the tenth dose. In aspects, only a pre-treatment time-covariate level is determined. In aspects, only a post-initial treatment time-covariate level is determined. In aspects, both a pre-treatment and post-initial treatment time-covariate level is determined.

The step of predicting a future concentration of the anti-TNFα biologic may include modeling the future level of an anti-TNFα biologic in the individual based on a time-varying covariate level in the individual and application of a correction factor to a population PK model for the therapeutic. For example, the correction factor may employ an Emax method as described herein. In aspects, the correction factor provides a predicted increase or decrease in the time-varying covariate following administration of the anti-TNFα biologic. Such increase or decrease takes into account the improvements that occur over time in the individual as a result of the anti-TNFα administration. In aspects, the Emax equation used for determining the correction factor is as follows:

$$Covariate values = E 0 * exp ( \frac{Emax * ( Dose Number - 1 )}{( D 50 - 1 ) + ( Dose Number - 1 )} )$$

wherein Emax is the maximum increase or decrease, dose number is the infliximab dose number (dose1, dose2, dose3 or dose4) and D50 is the dose number when the change was 50%. In aspects, D50 is fixed to 1.1.

In one aspect, the Emax for weight may be about 2 to about 7, or about 4 to about 5%, or +4.7%.

In one aspect, the Emax for serum albumin may about 8% to about 15%, or about 10% to about 12%, or +11.7%.

In one aspect, the Emax for erythrocyte sedimentation rate may be about −60% to about −65%, or about −62% to about −63%, or −62.4%.

In one aspect, the Emax for neutrophil CD64 may be about −20% to about −30%, or −25% to about −27%, or −26.5%.

The time-varying covariates may then be incorporated into a population PK model to account for the time-varying changes in covariate data from baseline during anti-TNFα treatment. For example, the population PK model may be described as follows:

$${C {L}_{ind} = C {L}_{p o p} \times WT / 65 )}^{0.594} \times {( ALB / 3.5 )}^{- 1.07} \times {( ESR / 9 )}^{0.101} \times {( nCD 64 / 4.6 )}^{0.168} \times {( ATI / 22 )}^{0.134} . V {1}_{ind} = V {1}_{pop} \times {( WT / 65 )}^{0.55} .$$

$$wherein {Q}_{ind} = {Q}_{pop} \times ( WT / 65 ) ;$$

$$wherein V {2}_{ind} = V {2}_{pop} \times {( WT / 65 )}^{0.586} ;$$

wherein ALB, albumin; ATI, antibody to infliximab; CI, confidence interval; CL, clearance; CV, coefficient of variation; ESR, erythrocyte sedimentation rate; Ind, individual; nCD64, neutrophil CD64 activity ratio; pop, population; PK, pharmacokinetic; Q, inter-compartmental clearance; V1, central compartment volume of distribution; V2, peripheral compartment volume of distribution.

Determination of one or more of weight, albumin, erythrocyte sedimentation rate (ESR), and neutrophil CD64 (nCD64) expression in an individual is within the skill of one of ordinary skill in the art, and employs known methods. Such determination of one or more of weight, albumin, erythrocyte sedimentation rate (ESR), and neutrophil CD64 (nCD64) expression may be made within a month of a first treatment of the individual with the biologic. In other aspects, the determination of one or more of weight, albumin, erythrocyte sedimentation rate (ESR), and neutrophil CD64 (nCD64) expression may be made within three weeks of treatment of the individual with the anti-TNFα biologic. In other aspects, the determination of one or more of weight, albumin, erythrocyte sedimentation rate (ESR), and neutrophil CD64 (nCD64) expression may be made within two weeks of treatment of the individual with the anti-TNFα biologic, within one week of treatment of the individual with the anti-TNFα biologic. In other aspects, the determination of one or more of weight, albumin, erythrocyte sedimentation rate (ESR), and neutrophil CD64 (nCD64) expression may be made within six days of treatment of the individual with the anti-TNFα biologic. In other aspects, the determination of one or more of weight, albumin, erythrocyte sedimentation rate (ESR), and neutrophil CD64 (nCD64) expression may be made within five days of treatment of the individual with the anti-TNFα biologic. In other aspects, the determination of one or more of weight, albumin, erythrocyte sedimentation rate (ESR), and neutrophil CD64 (nCD64) expression may be made within four days of treatment of the individual with the anti-TNFα biologic. In other aspects, the determination of one or more of weight, albumin, erythrocyte sedimentation rate (ESR), and neutrophil CD64 (nCD64) expression may be made within four days of treatment of the individual with the anti-TNFα biologic. In other aspect, the determination of one or more of weight, albumin, crythrocyte sedimentation rate (ESR), and neutrophil CD64 (nCD64) expression may be made within three days of treatment of the individual with the anti-TNFα biologic. In other aspects, the determination of one or more of weight, albumin, erythrocyte sedimentation rate (ESR), and neutrophil CD64 (nCD64) expression may be made within two days of treatment of the individual with the anti-TNFα biologic. In other aspects, the determination of one or more of weight, albumin, erythrocyte sedimentation rate (ESR), and neutrophil CD64 (nCD64) expression may be made within one month of treatment of the individual with the anti-TNFα biologic. In other aspects, the determination of one or more of weight, albumin, erythrocyte sedimentation rate (ESR), and neutrophil CD64 (nCD64) expression may be made the day of treatment of the individual with the anti-TNFα biologic.

In other aspects, the determination of one or more of weight, albumin, erythrocyte sedimentation rate (ESR), and neutrophil CD64 (nCD64) expression may be made following an initial treatment of the individual with the anti-TNFα biologic. In other aspects, the determination of one or more of weight, albumin, erythrocyte sedimentation rate (ESR), and neutrophil CD64 (nCD64) expression may be made within one day following treatment of the individual with the anti-TNFα biologic. In other aspect, the determination of one or more of weight, albumin, erythrocyte sedimentation rate (ESR), and neutrophil CD64 (nCD64) expression may be made within two days following treatment of the individual with the anti-TNFα biologic. In other aspects, the determination of one or more of weight, albumin, erythrocyte sedimentation rate (ESR), and neutrophil CD64 (nCD64) expression may be made within three days following treatment of the individual with the anti-TNFα biologic. In other aspects, the determination of one or more of weight, albumin, erythrocyte sedimentation rate (ESR), and neutrophil CD64 (nCD64) expression may be made within one week following treatment of the individual with the anti-TNFα biologic. In other aspects, the determination of one or more of weight, albumin, erythrocyte sedimentation rate (ESR), and neutrophil CD64 (nCD64) expression may be made within onc month following treatment of the individual with the anti-TNFα biologic. In other aspects, the determination of one or more of weight, albumin, erythrocyte sedimentation rate (ESR), and neutrophil CD64 (nCD64) expression may be made more than one month following treatment of the individual with the anti-TNFα biologic.

In aspects, the anti-TNFα biologic is an antibody. Tumor necrosis factor-alpha (TNF-( ) is a signaling protein involved in acute phase reaction and systemic inflammation. Therapeutic antibodies are known in the art and will be readily appreciated. In aspects, the anti-TNFα biologic is an antibody having a half-life of from about 5 to about 30 days, or from about 10 to about 20 days, or about 15 days. Exemplary anti-TNFα biologics include infliximab (known also as Remicade™), etanercept (Enbrel™), adalimumab (Humira™), certolizumab pegol (Cimzia™), and combinations thereof. The method of claim 1, the anti-TNFα biologic being infliximab.

At least one of the described steps may be carried out on a computer. For example, in one aspect, at least one of step (a), step (b), or step (c) is implemented using a computer. In one aspect, at least two of step (a), step (b), or step (c) is implemented using a computer. In one aspect, each of step (a), step (b), or step (c) is implemented using a computer.

The individual may be a pediatric patient or an adult. In one aspect, the individual is under 18 years of age. In one aspect, the individual is an adult.

The individual may be one who has not previously been exposed to an anti-TNFα therapeutic. For example, the individual may be anti-TNFα treatment naïve. Anti-TNFα treatment naïve may include an individual who has never been exposed to an anti-TNFα biologic. Anti-TNFα treatment naïve may include an individual who has not been exposed to an anti-TNFα biologic within one year, or six months, or five months, or four months, or three months, or two months, or one month.

In a further aspect, disclosed is a method of treating an individual having an inflammatory bowel disease (IBD), comprising
a. determining a value for one or more time-varying covariates selected from weight, serum albumin (ALB), erythrocyte sedimentation rate (ESR), and neutrophil CD64 ratio (nCD64) in the individual;
b. simulating a predicted concentration-time curve for the anti-TNFα biologic based on the biomarker value and a correction factor;
c. determining a dose of the anti-TNFα biologic based on the predicted concentration-time curve; and
d. administering to the individual, a dose of the anti-TNFα biologic.

The one or more time-varying covariates and detection thereof are as described herein. Likewise, the simulation of the predicted concentration-time curve may be determined based on the population PK model, with application of a correction factor, as described herein. In one aspect, the correction factor is the application of the Emax model as described herein. The dose determined may be one or both of an initial dose and a subsequent dose administered following the initial dose. In other aspect, the dose may be one administered during an induction phase, as described herein. In other aspect, the dose may be one administered during a maintenance phase, as described herein.

Administration of the anti-TNFα biologic based on the predicted concentration-time curve may be carried out via any suitable route, for example, via intravenous (IV) administration. In one aspect, the anti-TNFα biologic is infliximab and is administered intravenously to the individual, in an amount determined based on the predicted concentration-time curve. In one aspect, the anti-TNFα biologic is etanercept and is administered intravenously to the individual, in an amount determined based on the predicted concentration-time curve. In one aspect, the anti-TNFα biologic is adalimumab and is administered by subcutaneous injection to the individual, in an amount determined based on the predicted concentration-time curve. In one aspect, the anti-TNFα biologic is certolizumab pegol and is administered by subcutaneous injection to the individual, in an amount determined based on the predicted concentration-time curve.

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1

Current iterations of infliximab pharmacokinetic models, do not account for expected improvements in covariates of drug clearance, leading to inaccurate predictions of drug concentrations and imprecise starting doses. Applicant aimed to identify changes in four biomarkers associated with infliximab clearance (Xiong et al. model) and determine if integration of these dynamic changes would improve model performance during the induction period. Applicant found that use of time varying covariates with a correction factor, such as using an Emax time-varying covariates analytic approach, was superior to use of standard modeling procedures.

Applicant analyzed two cohorts of children receiving infliximab for Crohn's Disease. The Emax method was used to assess time-varying changes in covariates. Model performance (observed vs. predicted infliximab concentrations) was evaluated using median percentage error (bias) and median absolute percentage error (precision). The combined cohorts included 239 Crohn's disease patients. Applicant found from baseline to dose4, the Emax for weight was 0.046 (+4.7%), Emax for serum albumin was 0.110 (+11.7%), Emax for erythrocyte sedimentation rate was −0.978 (−62.4%), and the Emax for neutrophil CD64 was −0.362 (−26.5%). Furthermore, when these four time-varying covariates with advanced modeling were applied to predict future infliximab concentrations, Applicant found there was no statistical differences between the median observed and predicted concentrations. Applicant found that integrating advanced modeling into pharmacokinetic modeling improves precision during the induction phase of infliximab in patients with Crohn's disease. These results suggest that novel modeling strategies during the induction dose selection process may lead to improved drug exposure.

In order to better characterize infliximab pharmacokinetics (PK) in children, Applicant developed a population PK model for infliximab based on real-world data obtained from children and young adults with CD.¹⁰ Subsequently, a multidisciplinary team created an electronic health record (EHR) integrated model informed precision dosing (MIPD) platform to standardize anti-TNFα dose optimization strategies during the induction and maintenance phases for both children and adults with IBD.¹¹ As part of the development of this population PK model and dosing platform, Applicant discovered model accuracy improved when the five covariates of drug clearance (serum albumin, patient weight, erythrocyte sedimentation rate [ESR], antidrug antibody (ADA) and the neutrophil CD64 [nCD64] ratio) were included.¹⁰ However, as this population PK model was developed from the collection of both induction and maintenance infliximab concentrations, it was believed that model imprecision during the induction phase would occur given the dynamic changes in the covariates of drug clearance when a patient is healing (responding to treatment).

Time-varying covariate modeling in pharmacometrics can predict and quantify the PK course over time utilizing biomarkers, disease phenotypes or clinical symptoms.¹² It is believed the effect of dynamic changes in the covariates, reflecting early treatment response, on the accuracy of predictions and its integration as a standard procedure to optimize biologic exposure during the induction phase, prior to Applicant's invention, has not been described in the IBD literature. Applicant sought to identify the expected changes in the biomarkers of infliximab clearance and evaluate whether integration of predicted early response in the time-varying covariates in the induction phase of infliximab dosing would provide more precise drug concentration predictions during the early (dose3) and the immediate post-induction period (dose4).

### Methods

Study population. Two cohorts of children and young adults receiving infliximab for CD were included in this investigation. The Targeting the Inflammatory Signature to Personalize Biologics in Pediatric IBD (REFINE) study was a multicenter, observational cohort that prospectively enrolled 78 children and young adults starting infliximab for CD at four medical centers from 2014-2019. The REFINE eligibility criteria has been reported in prior investigations.^{10,13} During the REFINE study, longitudinal biomarkers of inflammation and infliximab concentrations were determined throughout the first year of infliximab therapy. The Accuracy of a PK Physician Decision Support Dashboard for Children receiving Infliximab for IBD (APPDASH) cohort included 161 children and young adults with CD who started infliximab at Cincinnati Children's Hospital Medical Center (CCHMC) from 2019-2021. Inclusion criteria for this retrospective cohort included patients 2-22 years old with luminal CD who had received a minimum of four infliximab infusions at CCHMC and had at least one infliximab TDM performed prior to receiving a fourth infliximab infusion (dose4). Those with UC or IBD-unspecified, those with internal penetrating CD or patients with missing infusion data were excluded. The EHR was queried to collect infliximab dosing (mg), infusion dates, start and stop times, patient weight, height, and all laboratory data associated with each infusion. With both cohorts, Applicant recorded patient demographics, CD extent and severity using the Paris Classification¹⁴ and verified all past and current CD treatments.

Trough concentrations. In the REFINE cohort, the trough concentration was obtained at every infusion for the first year (including a post-infusion peak after dose 1, 3, and 4). At minimum, the APPDASH cohort had a trough concentration obtained at either dose3 or dose4. Infliximab and ADA concentrations for both cohorts were measured with the electrochemiluminescence immunoassay (ECLIA; Esoterix, LabCorp Specialty lab, Calabasas, CA). The lower limit of detection for infliximab is 0.4 μg/mL and 22 ng/mL for ADA. Any value below the limit of infliximab detection was removed from the analysis.

Biomarkers of infliximab clearance. The laboratory results of interest included serum albumin (g/dL), ESR (mm/h), c-reactive protein (CRP; mg/dL), nCD64 (ratio) and the fecal calprotectin (μg/g).

Study outcomes. Clinical remission was defined as a weighted pediatric CD activity index (wPCDAI) 12.5 and off prednisone at dose4. Clinical remission was only determined in the REFINE cohort as there was no reliable method to assess for wPCDAI remission with the retrospective APPDASH cohort.

Statistical analysis. Continuous variables are represented as means with standard deviations (SD) or medians with 25-75% interquartile range (IQR) depending on data distribution. The Wilcoxon signed-rank test was used to compare paired groups. The Friedman test, followed by Wilcoxon signed rank test with Bonferroni correction, was used to compare nonparametric paired data across more than three groups. PK analysis and simulations were performed by nonlinear mixed-effects modeling (NONMEM) software (version 7.5.1; ICON Development Solutions, Ellicott City, MD). R software version 4.1.0 (https://www.R-project.org) was used for graphical and statistical analysis.

Estimate time-varying changes in covariate data. Applicant hypothesized that a mathematical model that accounted for improvements in the biomarkers of infliximab clearance would improve the prediction of infliximab trough concentrations (precision and accuracy) compared to a method of using the baseline covariates alone. Therefore, the stepwise approach was to describe the time-varying changes in the four biomarkers of infliximab clearance and quantify these changes as the delta percentage from baseline to the specific timepoint of interest. First, Applicant compared the percent change in each of the four covariates (albumin, weight, ESR, and nCD64) from baseline (pretreatment) to dose3 (week6) and from baseline to dose4 (˜week 14).

Secondly, in order to incorporate time-varying changes in the covariates into the previously published population PK model¹⁰, Applicant described covariate data changes over the course of treatment using the Emax model. The Emax equation is below:

$$Covariate values = E 0 * exp ( \frac{Emax * ( Dose Number - 1 )}{( D 50 - 1 ) + ( Dose Number - 1 )} )$$

In this equation, E0 is the baseline covariate value, Emax is the maximum increase or decrease, dose number is the infliximab dose number (dose1, dose2, dose3 or dose4) and D50 is the dose number when the change was 50%. In this analysis, D50 could not be estimated due to the lack of information on time-course covariate changes between doses. Therefore, D50 was fixed to 1.1 to describe the most drastic covariate changes observed between dose1 and dose2 in this study. The Emax parameters were estimated using the Im4 R package (version 1.1-32). The random effect of between-subject variability to E0 was considered in the estimation of the parameters.

### Evaluation of Model Prediction

The time-varying parameters were then incorporated into the previously published population PK model¹⁰ to account for the dynamicchanges in covariate data from baseline during infliximab treatment. The population PK model is as follows:

$${C {L}_{ind} = C {L}_{p o p} \times WT / 65 )}^{0.594} \times {( ALB / 3.5 )}^{- 1.07} \times {( ESR / 9 )}^{0.101} \times {( nCD 64 / 4.6 )}^{0.168} \times {( ATI / 22 )}^{0.134} . V {1}_{ind} = V {1}_{pop} \times {( WT / 65 )}^{0.55} .$$

$${Q}_{ind} = {Q}_{pop} \times ( WT / 65 ) . V {2}_{ind} = V {2}_{pop} \times {( WT / 65 )}^{0.586} .$$

Abbreviations: ALB, albumin; ATI, antibody to infliximab; CI, confidence interval; CL, clearance; CV, coefficient of variation; ESR, erythrocyte sedimentation rate; Ind, individual; nCD64, neutrophil CD64 activity ratio; pop, population; PK, pharmacokinetic; Q, inter-compartmental clearance; RSE, relative standard error; V1, central compartment volume of distribution; V2, peripheral compartment volume of distribution; wPCDAI, weighted pediatric CD activity index.

Applicant assessed the effect the time-varying changes in covariate data had on model prediction for the infliximab concentration as compared to the observed (actual) infliximab concentration. Additionally, the predictive performance of the infliximab trough concentration was evaluated using available clinical data (observed infliximab concentration and covariate data) for each dose by considering realistic clinical situations (i.e., clinical data availability). Bayesian estimation^{15,16} was used to predict the trough infliximab concentration if the observed infliximab concentration was available. The bias and precision of the predictions were evaluated using the median percentage error (MPE) and median absolute percentage error (MAPE), respectively, as follows.

$$Median percentage error ( MPE ) = Median \frac{{C}_{pred} - {C}_{obs}}{{C}_{obs}} \times 100$$

$$Median absolute percentage error ( MAPE ) = Median \vert \frac{{C}_{pred} - {C}_{obs}}{{C}_{obs}} \vert \times 100$$

C_{pred} is the predicted infliximab trough concentration and C_{obs} is the observed infliximab trough concentration.

### Results:

Patients. In total, the REFINE and APPDASH cohorts included 239 patients with CD. The median (IQR) age at the start of infliximab was 14.3 (11-16) years, 40.6% were female, and 86.6% were white. The median starting dose was 7.1 (5.5-10) mg/kg with additional demographic data presented in Table 1. There were notable differences in the median time to start infliximab from diagnosis, the starting dose, age at diagnosis, weight at infliximab start and the nCD64 ratio between the two cohorts.

TABLE 1
Patient demographics and baseline disease characteristics. All values are listed
as the median (25%-75% interquartile range) or number (n) with percentage (%).

| | Combined Cohorts | | REFINE | | APPDASH | |
|---|---|---|---|---|---|---|
| Characteristic | (n = 239) | | (n = 78) | | (n = 161) | |
| Age at diagnosis, years | 13.9 | (10.1-16) | 12.7 | (9.7-15.4) | 14.3 | (11.1-16.5)* |
| Age at first infusion, years | 14.3 | (11-16.3) | 13.2 | (10.6-15.9) | 14.6 | (11.5-16.7) |
| Female, n (%) | 97 | (40.6%) | 28 | (35.9%) | 69 | (42.9%) |
| White race, n (%) | 207 | (86.6%) | 72 | (92.3%) | 135 | (83.9) |
| Time to start infliximab, days | 37 | (15-112) | 49 | (18-382) | 35 | (10-80)** |
| Infliximab starting dose, mg/kg | 7.1 | (5.5-9.9) | 6.1 | (5.2-7.1) | 9 | (5.8-10)0*** |
| Crohn's location (L1:L2:L3) | 31:46:162 | | 9:7:62 | | 22:39:100 | |
| Crohn's behavior (B1:B2:B3:B4) | 186:26:18:9 | | 66:7:4:1 | | 120:19:14:8 | |
| Perianal phenotype, n (%) | 30 | (12.6%) | 12 | (15.4%) | 18 | (11.2%) |
| Weight at first infusion, kg | 47 | (32.4-60.9) | 41 | (28-56.8) | 49.4 | (34.5-61.8)* |
| Serum albumin, g/dL | 3.4 | (2.9-3.8) | 3.4 | (3-3.8) | 3.4 | (2.9-3.8) |
| Erythrocyte sedimentation rate, mm/h | 16 | (9-35) | 15 | (8-38) | 16 | (9-35) |
| Neutrophil CD64 activity ratio | 6.4 | (5.9-7.1) | 6.5 | (6-7.1) | 4.5 | (3.8-10.8)* |

*p < 0.05;
**p < 0.01;
***p < 0.001.
L1, ileum only; L2, colon only; L3, ileocolonic; B1, inflammatory; B2, stricturing; B3, penetrating; B4, both stricturing and penetrating.

Estimate time-varying changes in covariate data. In a prior population PK study, Xiong et al. identified five biomarkers of infliximab clearance (patient weight, serum albumin, ESR, ADA and nCD64) that improved the model performance.¹⁰ Combining the data from both cohorts, Applicant evaluated the observed percent improvement (delta) in four covariates (weight, albumin, ESR and nCD64) from baseline to dose3 and from baseline to dose4. Applicant also identified the percent improvement for the subset of patients who (a) had a dose3 trough concentration ≥18 μg/mL, (b) had a dose4 trough concentration ≥5 μg/mL, and (c) achieved clinical remission at dose4 (Table 2).

TABLE 2
The observed improvement in the four covariates of
drug clearance from dose1-dose3 and dose1-dose4.

| Timepoint/Outcome | Δ weight | Δ albumin | Δ ESR | Δ nCD64 |
|---|---|---|---|---|
| A. Dose3 (all subjects) | 6% | 16% | −41% | −25% |
| B. Dose3 ≥18 μg/mL | 9% | 17% | −21% | −17% |
| C. Dose4 (all subjects) | 8% | 16% | −25% | −15% |
| D. Dose4 ≥5 μg/mL | 10% | 15% | −33% | −26% |
| E. Dose4 clinical | 10% | 17% | −25% | −36% |
| remission | | | | |
| Mean (standard | 8.6% | 16% | −29% | −24% |
| deviation) | (1.7) | (0.84) | (8) | (8.4) |

Dose3, week 6 infusion; dose4, first maintenance infusion; ESR, erythrocyte sedimentation rate; nCD64, neutrophil CD64 ratio; clinical remission based on a weight pediatric Crohn's disease index <12.5 and off steroids at dose4. Clinical remission was assessed in the REFINE cohort (n = 78) only.

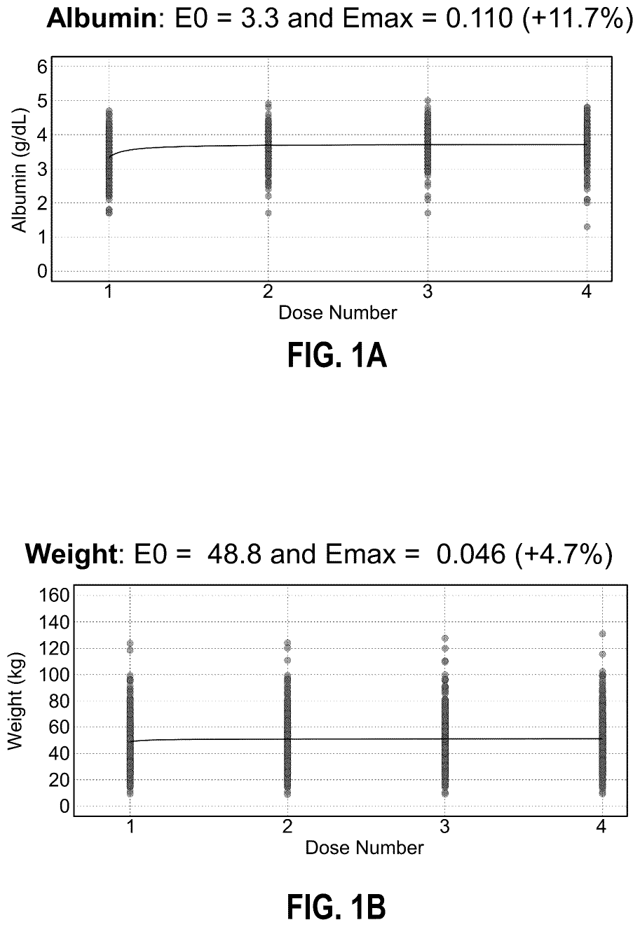

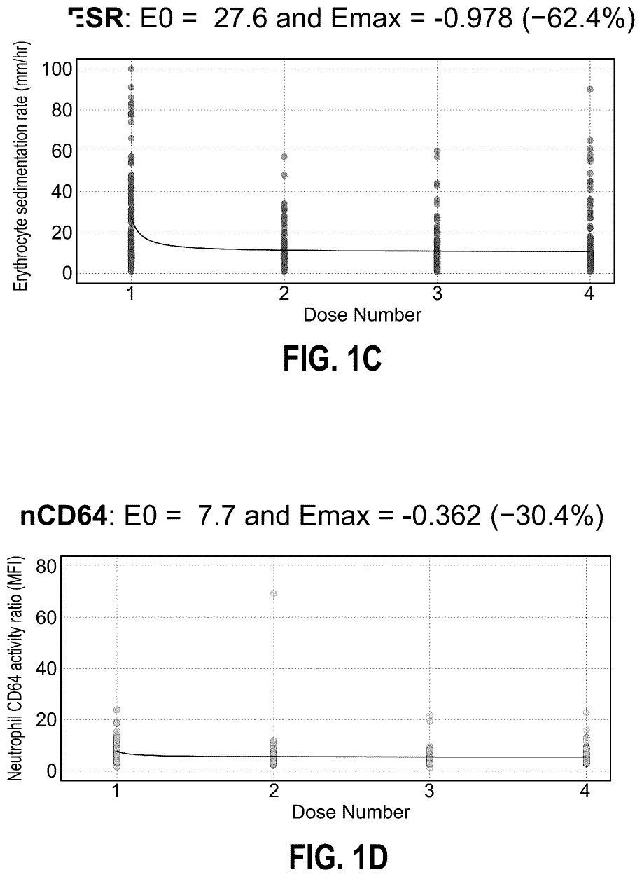

In a subsequent analysis, Applicant sought to estimate the time-varying changes in the four covariates using the Emax method. Using the Emax model allows for estimating the mean changes of covariate data after starting infliximab treatment and incorporating the time vary changing into the population PK model as Emax functions. From baseline to dose4 using the combined cohorts, Applicant found the Emax for weight was 0.046 (+4.7%), Emax for serum albumin was 0.110 (+11.7%), Emax for ESR was −0.978 (−62.4%), and the Emax for nCD64 was −0.362 (−26.5%, FIG. 1).

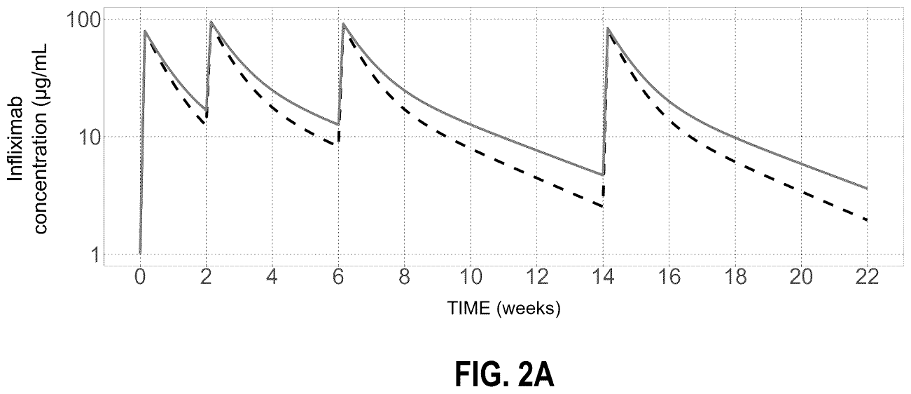

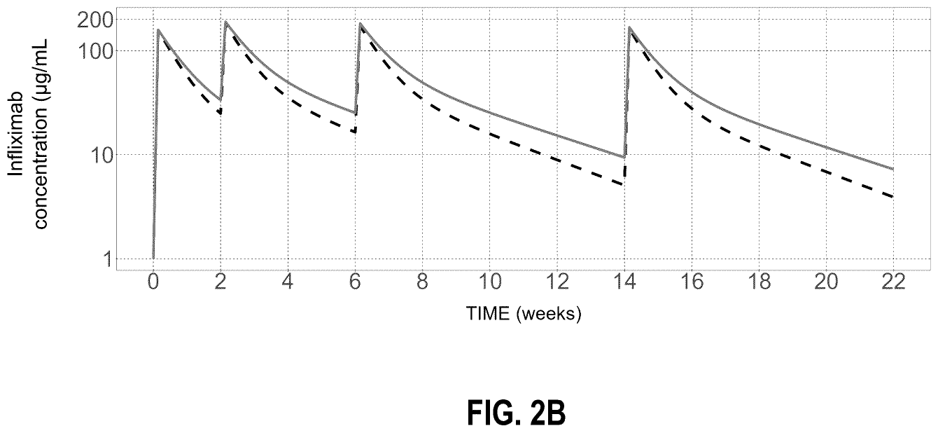

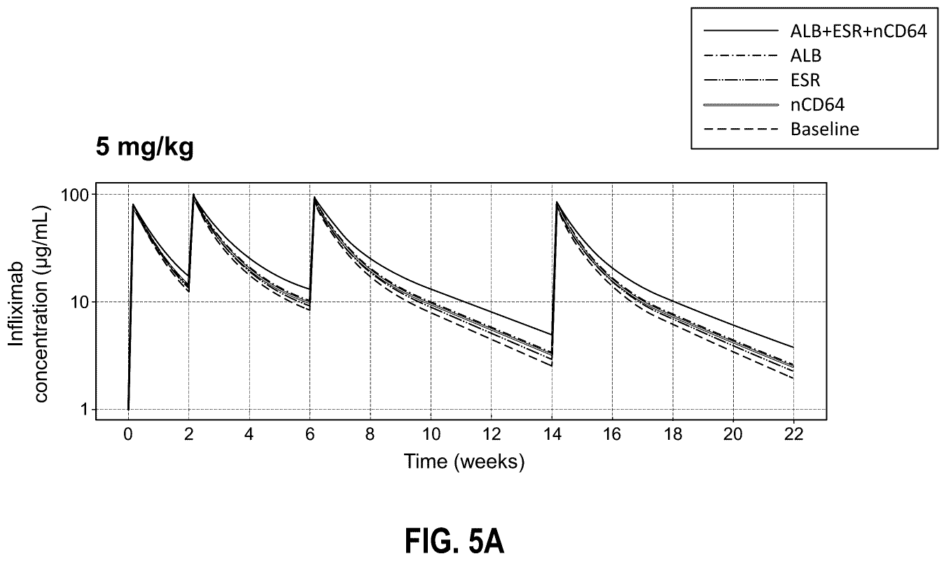

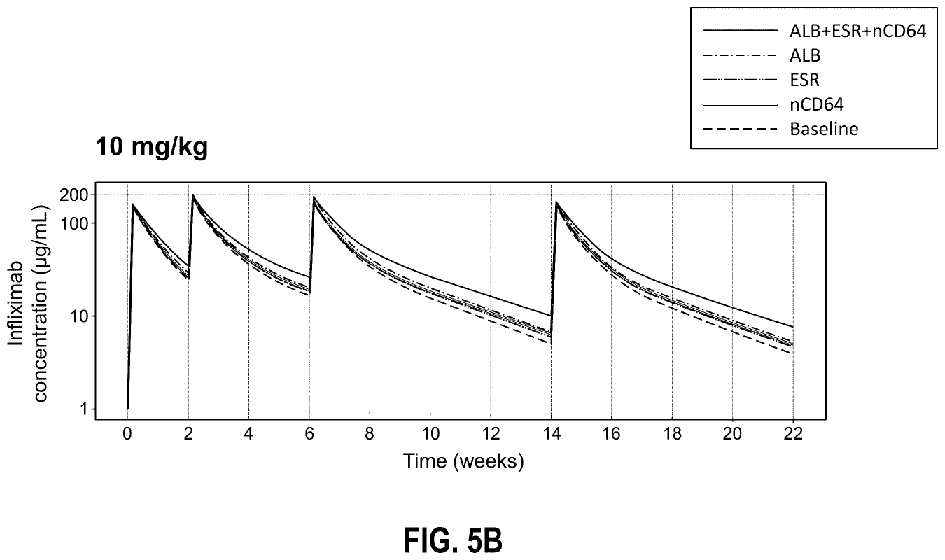

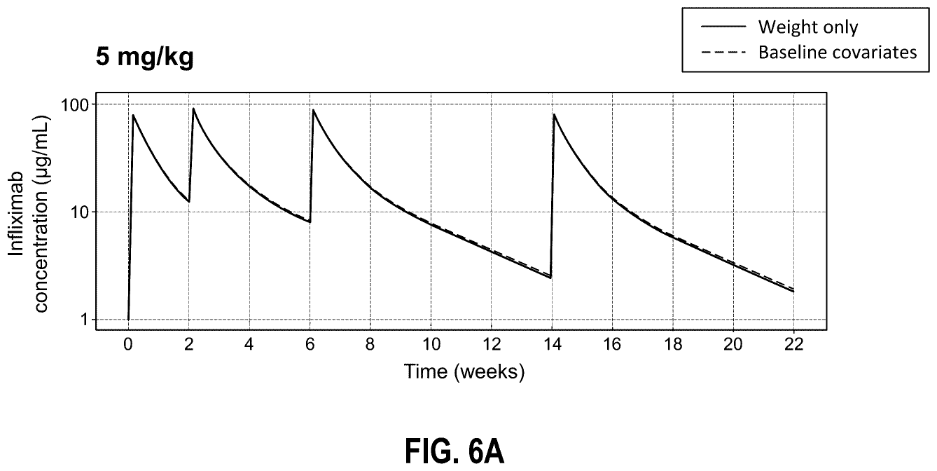

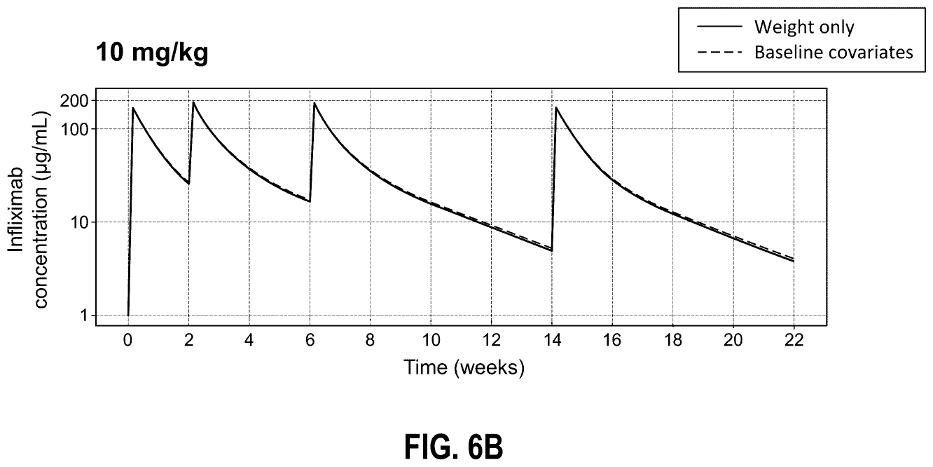

The infliximab PK profiles for a 47 kg (median body weight) patient receiving 5 mg/kg or 10 mg/kg with the inclusion of the Emax calculated time-varying covariates and compared these to simulations without considering dynamic covariate changes (calculations with baseline covariates only). Whether starting at 5 or 10 mg/kg, the infliximab trough concentrations were predicted to be higher at each timepoint when all four covariates (weight, albumin, ESR and nCD64) with the predicted (time-varying changes) were applied (FIG. 2). Applicant also found that the predicted infliximab trough concentrations were consistently predicted to be higher whether one, two or all three covariates (albumin, ESR, and nCD64) were included (FIG. 5). Interestingly, the inclusion of weight alone as a time-varying covariate did not predict a higher infliximab concentration for either the 5 or 10 mg/kg simulations (FIG. 6).

### Evaluation of Model Prediction

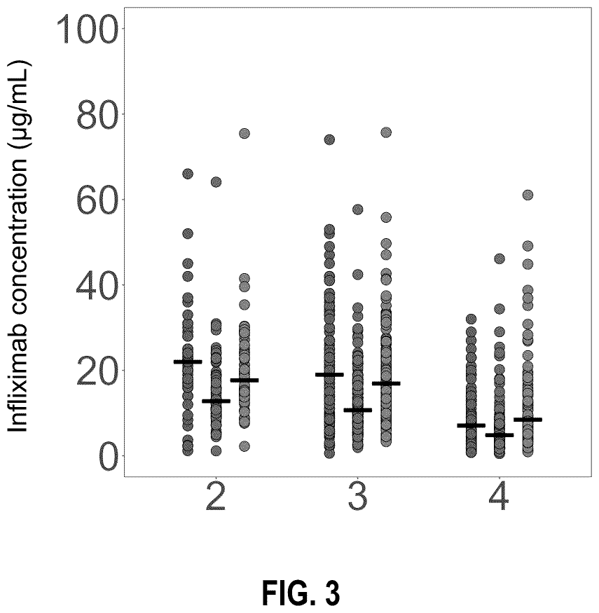

Following identification of the mean change in covariates and performing of dosing simulations, Applicant sought to evaluate model precision and accuracy by assessing the median differences in the observed and predicted infliximab trough concentrations. Using only the baseline covariates and the Xiong et al. population model,¹⁰, Applicant found that the median predicted infliximab concentrations at dose2, dose and dose4 were lower than the median observed concentrations (FIG. 3). Incorporating time-varying covariate changes with Emaxmodeling, however, resulted in comparable infliximab median concentrations between the observed and predicted (there was no statistical difference at any timepoint).

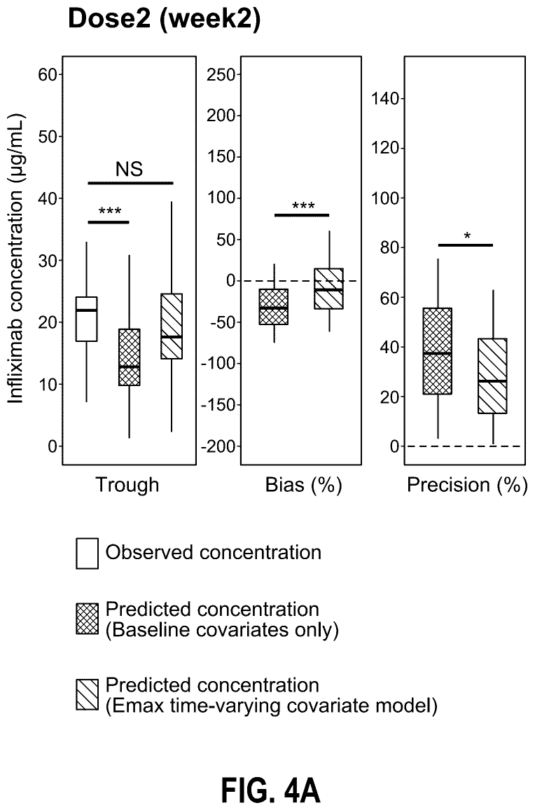

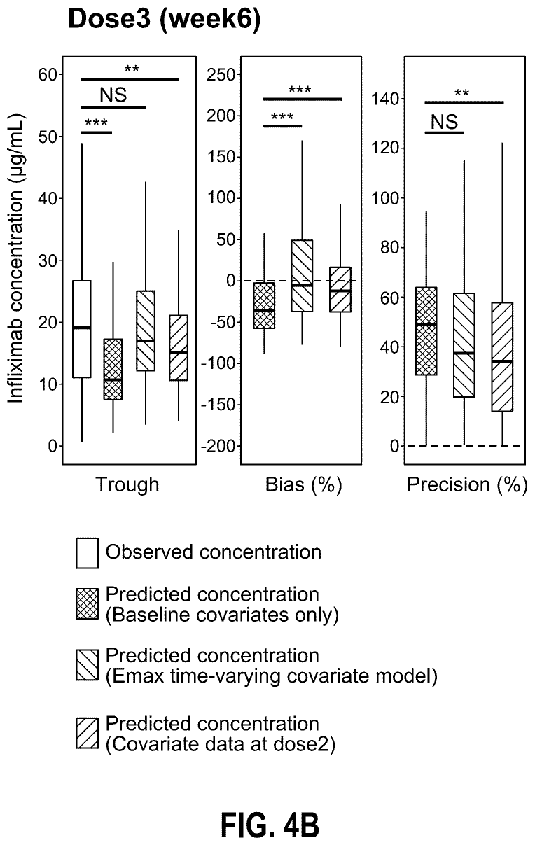

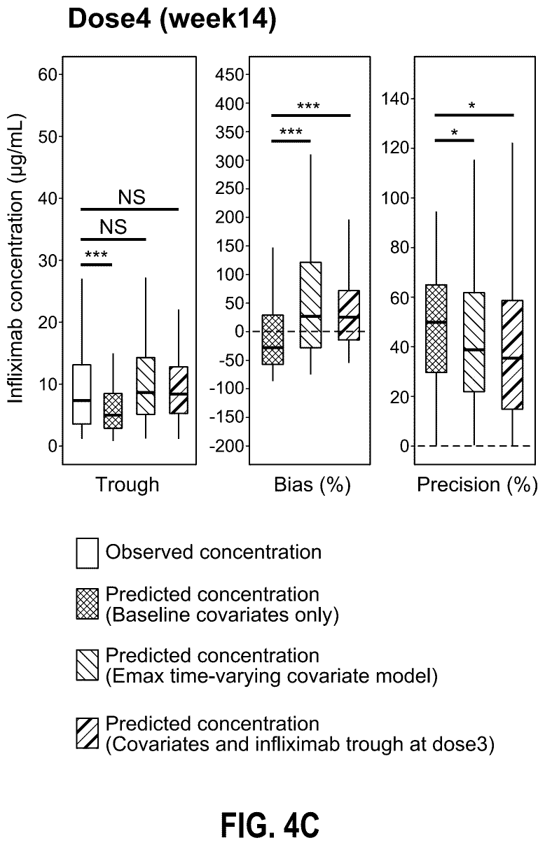

Model predictive performance for the next infliximab trough under real-world conditions was evaluated. For example, to predict a dose2 infliximab trough, the model must rely on the baseline (pre-treatment) covariates alone. Similarly, to predict dose3 infliximab trough, the model can rely on the observed covariates at dose1 and/or dose2 (therapeutic drug monitoring (TDM) is not commonly performed at dose2). Yet, to predict a dose4 infliximab trough, the model can rely on both the covariates (at doses 1, 2, and 3) and the infliximab trough concentration at dose3 (week6). In fact, proactive TDM at dose3 has become more common in real-world clinical practice, especially in patients anticipated to have more rapid drug clearance.⁹ Based on these real-world scenarios, Applicant assessed the model performance to predict the next infliximab trough concentration using the prior covariates and infliximab trough concentrations (if available) at the last infusion. Applicant then assessed bias and precision to predict a dose2 infliximab trough concentration using (a) the baseline covariates only and (b) with the Emax time varying covariates. Applicant found that the bias and precision were improved when the Emax time varying model was used (FIG. 4, panel a). The comparison of dose3 trough concentrations predictions were performed with the following conditions: (a) with baseline covariates only, (b) with Emax time varying covariates, and (c) the observed covariates at dose2. Applicant found the observed and predicted trough concentrations using the Emax method were similar (FIG. 4, panel b). Applicant also found predictions using the baseline covariates alone to forecast the dose3 trough concentration were inferior to the Emax method with a significant improvement in bias and a mild (non-significant) improvement in precision.

Applicant evaluated dose4 trough concentration predictions with the following scenarios: (a) baseline covariates alone, (b) time-varying covariates only (without TDM), and (c) the observed dose3 covariates and dose3 measured trough concentrations. Applicant found that the use of baseline covariates alone significantly underestimated the trough concentrations at dose4 (FIG. 4, panel c). Moreover, Applicant found that the bias and precision were significantly improved when the time-varying covariate prediction model or Bayesian estimation using the observed covariate and dose3 trough concentrations were used. The more detailed results for evaluating the prediction performance are summarized in Table 3.

TABLE 3
Overall model predictive performance with and without time-varying covariate analysis. Concentrations
are presented as median values. Statistical significance was determined by comparing scenario
b (or scenario c) to scenario a (baseline covariates) for each of the three doses.

| | | Data | | | | | |
|---|---|---|---|---|---|---|---|
| | | included for | | Observed | Predicted | | |
| | | trough | Number | concentration | concentration | | Precision |
| Dose | Scenario | prediction | of obs. | (μg/mL) | (μg/mL) | Bias (%) | (%) |
| 2 | a | Baseline | 63 | 22 | 12.8 | −34.1 | 37.5 |
| | | covariates | | | | | |
| 2 | b | Time-varying | 63 | 22 | 17.7 | −12.2*** | 26.4* |
| | | covariates | | | | | |
| 3 | a | Baseline | 131 | 19 | 10.7 | −37.1 | 49.1 |
| | | covariates | | | | | |
| 3 | b | Time-varying | 131 | 19 | 16.9 | −5.2*** | 37.8 |
| | | covariates | | | | | |
| 3 | c | Covariates | 131 | 19 | 15 | −12.3*** | 34.5** |
| | | at dose2 | | | | | |
| 4 | a | Baseline | 94 | 7.1 | 4.9 | −28.4 | 58.9 |
| | | covariates | | | | | |
| 4 | b | Time-varying | 94 | 7.1 | 8.5 | 24.9*** | 48.5* |
| | | covariate | | | | | |
| 4 | c | Covariates | 94 | 7.1 | 8.3 | 22.4*** | 37.4* |
| | | at dose3 and | | | | | |
| | | infliximab | | | | | |
| | | trough at | | | | | |
| | | dose3 | | | | | |
| | | (Bayesian) | | | | | |

Obs, observations; *, p < 0.05; **, p < 0.01; ***, p < 0.001.

### DISCUSSION

While the FDA and EMA approved dose for infliximab is 5 mg/kg, contemporary real-world studies have shown induction doses for children with IBD often vary between 5-10 mg/kg.^{10, 17} Therefore, given the variability in dose selection and the complexity of predicting infliximab clearance in patients with IBD, this study was designed to assess the changes in the biomarkers of infliximab clearance during induction to standardize dose selection in real-world practice.

Currently, without regular access to decision support tools or PK modeling software, clinicians rely on a trial-and-error based approach for the initial dose selection and TDM to personalize infliximab maintenance regimens. More advanced strategies to guide dose selection or optimization include the use of covariates of infliximab clearance such as weight, serum albumin, presence of ADA and the inflammatory biomarkers such as ESR, nCD64 and CRP.^{10, 18-20} Additional studies have identified several patient and disease specific predictors associated with more rapid clearance and include age <10 years old, weight <30 kg, a low body mass index (BMI) and the extent and severity of gut injury.^{10, 13, 21}

Selecting the initial dose based on pretreatment covariates of drug clearance may not vary significantly for a therapeutic compound with a short half-life. However, it poses several challenges for a therapeutic with a long half-life (such as infliximab that varies between 12.4-18.8 days^{10,18}) and when the PK is dependent on the disease process itself, including antigen burden and a leaky gut.²² Applicant therefore, considered predicting early improvements in infliximab clearance covariates, reflecting on treatment response, and its integration into the PK model as a method to maximize the initial exposure, safety, and costs of care while also striving to minimize over-exposure and delays in third party approval. In clinical and systems pharmacology, disease progression models are integrated in PK/pharmacodynamic models to account for the influence of drug treatment on disease progression or response over time.¹² Integrating this novel approach to manage IBD is necessary for advanced therapies given the complex interactions between disease severity, inflammatory biomarkers, and drug exposure²² with acute severe ulcerative colitis serving as the prototypical trial-and-error example and the difficulty this causes in selecting the correct starting dose.²⁰ In fact, in a cross sectional study of patients with CD undergoing colonoscopy while receiving infliximab, Applicant found significant increases in drug clearance in patients with endoscopically active CD as compared to those with achieved endoscopic healing.²³

Infliximab induction (75-88.4%) has a high response rate^{10,24,25}, patient weight and serum albumin improved, and the inflammatory biomarkers (ESR and nCD64) decreased during the early induction period. Whether Applicant used the delta improvement approach or the Emax method, Applicant not only found the biomarkers improved but found that use of the Emax method to predict early (dose3 and dose4) infliximab concentrations was superior to use of the Xiong et al. population PK model¹⁰ alone (relying only on baseline covariates). Interestingly, utilizing singular covariates (such as weight only) to simulate trough concentrations was consistently shown to predict a lower infliximab exposure as compared to using a combination of the biomarkers to predict future infliximab concentrations. Therefore, the potential consequences of not considering the effect of early treatment response on infliximab pharmacokinetic covariates when selecting a starting dose is over-estimating the dose (higher) needed to achieve a target concentration, which may delay third-party drug approval and unnecessarily increase healthcare costs.

This study benefited from the considerable number of children and young adults that were included and the large number of infliximab concentrations obtained using the same laboratory (Esoterix, LabCorp). Applicant applied straightforward statistical approaches to identify the expected improvements in the biomarkers of drug clearance and utilized these identified improvements to inform our baseline population PK model. Except for the nCD64 test, all other biomarkers included in the model are easy to obtain in any clinical setting. Furthermore, Applicant clearly demonstrated that use of weight alone was insufficient to accurately predict infliximab exposure during induction.

The variability between the two cohorts (differences in the time to start infliximab and starting dose) reflect the transformation in infliximab dosing, especially in pediatric IBD and the importance of identifying a more uniform approach to dosing infliximab. Study limitations included the inability to retrospectively track clinical outcomes in the APPDASH cohort and the lack of more objective measures, such as endoscopy, in both cohorts. While fecal calprotectin was obtained in the REFINE cohort for some participants, Applicant did not look to define delta differences for this biomarker given the missingness of fecal calprotectin data in both cohorts. Additionally, while the REFINE cohort had TDM performed throughout induction, TDM use for the APPDASH cohort was largely at the discretion of the clinician.

Although the use of dosing platforms is not universal, there remains a limited number of advanced therapies approved for children. Several studies have indicated that employing the standard infliximab dosing of 5 mg/kg is linked to trough concentrations below the desired target concentrations.^{10,17,26}

### Example 2

The disclosed methods may be used to select an initial dose of an anti-TNFα biologic for administrating to an individual in need thereof. For example, a correction factor, in which the percent improvement of a covariate is incorporated into a population PK model, allows for selection of an appropriate starting dose of the therapeutic which, based on a predicted concentration-time curve, increases the likelihood of administering an effective dose which is not sub-therapeutic or in excess of a therapeutic dose. As a result of the correction factor applied to the model, an initial dose may be selected based on a pre-treatment covariate which accounts for the expected improvement of the covariates over time. Alternatively, or in addition, the correction factor may be applied to the model for determination of a subsequent dose based on a covariate level at the time of a given dose, wherein the next or subsequent dose can be determined, taking into account the expected improvement of the covariates over time. In particular, the correction factor may be applied to the model for determination of a dose administered during the induction or maintenance phase of the treatment. As a result, treatment selection is optimized, avoiding over- or under-dosing.

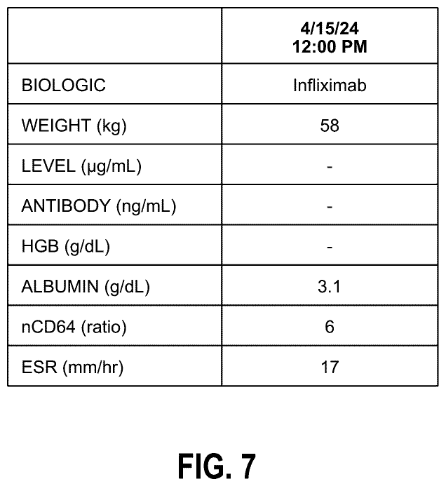

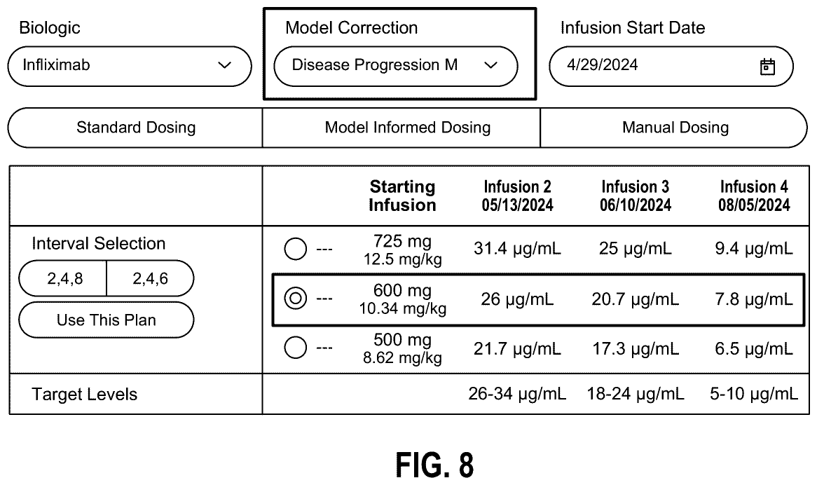

FIGS. 7-9 depict exemplary interfaces of a system employing the described methods. For example, an individual (typically, a patient) is initially diagnosed with an inflammatory condition such as IBD, Crohn's disease, ulcerative colitis, uveitis, rheumatoid arthritis, or the like, which generally requires the use of infliximab, a biosimilar, or other biologic as described herein. A blood draw is performed prior to treatment with a biologic, and laboratory tests to assay the covariates of biologic clearance (albumin, erythrocyte sedimentation rate, neutrophil CD64) are carried out. Weight is recorded. The covariates are added to the system. FIG. 7 depicts a screen shot of exemplary inputs of weight, albumin, nCD64, and ESR. After inputting the covariate data, the predicted percent improvement for each of the covariates are applied, for example, using the Emax method as described herein. The percent improvement values are used in the model. FIG. 8 depicts an exemplary interface showing the output of the starting dose with the model correction applied (“disease progression”). As can be seen in FIG. 8, the model suggests a 600 mg dose based on the model with the correction applied. Based on the model predictions, the patient is administered a 600 mg dose. The adjusted model prediction allows for treatment of the patient with the correct dose throughout the induction period (short term) and the maintenance period (long-term use).

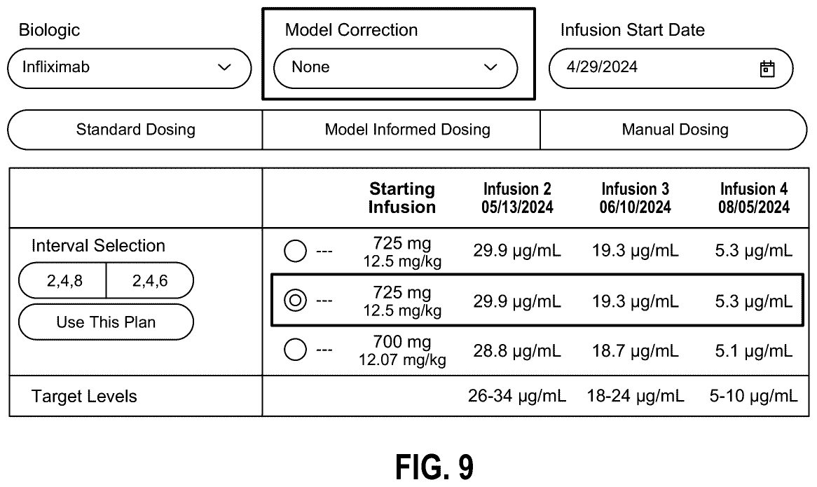

FIG. 9 depicts an example interface in which the model correction is not applied. FIG. 9 shows that, for example, without the correction, the Xiong et al. model would have indicated a higher dose of 725 mg. Thus, using the personalized dosing, which combines the model correction with the Xiong et al. model, allows for accurate dosing of 600 mg, avoiding immunosuppression which may occur with a higher than necessary dose.
1. Rosen M J, Dhawan A, Saced S A. Inflammatory Bowel Disease in Children and Adolescents. JAMA Pediatr. November 2015; 169 (11): 1053-60. doi: 10.1001/jamapediatrics.2015.1982
2. Kuenzig M E, Fung S G, Marderfeld L, et al. Twenty-first Century Trends in the Global Epidemiology of Pediatric-Onset Inflammatory Bowel Disease: Systematic Review. Gastroenterology. April 2022; 162 (4): 1147-1159.c4. doi: 10.1053/j.gastro.2021.12.282
3. Herzog D, Fournier N, Buchr P, et al. Prevalence of intestinal complications in inflammatory bowel disease: a comparison between paediatric-onset and adult-onset patients. Eur J Gastroenterol Hepatol. August 2017; 29 (8): 926-931. doi: 10.1097/MEG.0000000000000896
4. Church P C, Hyams J, Ruemmele F, de Ridder L, Turner D, Griffiths A M. The Continental Divide: Anti-TNF Use in Pediatric IBD Is Different in North America Compared to Other Parts of the World. Can J Gastroenterol Hepatol. 2018; 2018:3190548. doi: 10.1155/2018/3190548
5. Minar P, Saced S A, Afreen M, Kim M O, Denson L A. Practical Use of Infliximab Concentration Monitoring in Pediatric Crohn Disease. Journal of pediatric gastroenterology and nutrition. May 2016; 62 (5): 715-22. doi: 10.1097/MPG.0000000000001029
6. Assa A, Matar M, Turner D, et al. Proactive Monitoring of Adalimumab Trough Concentration Associated With Increased Clinical Remission in Children With Crohn's Disease Compared With Reactive Monitoring. Gastroenterology. October 2019; 157 (4): 985-996.e2. doi: 10.1053/j.gastro.2019.06.003
7. Holland K, Bennett W E, Slaven J E, Collier J, Waltz G, Pfefferkorn M. Proactive measurement of infliximab drug levels in children with Crohn's disease. Ann Gastroenterol. 2022; 35 (1): 56-62. doi: 10.20524/aog.2021.0676
8. van Hoeve K, Dreesen E, Hoffman I, et al. Adequate Infliximab Exposure During Induction Predicts Remission in Paediatric Patients With Inflammatory Bowel Disease. Journal of pediatric gastroenterology and nutrition. June 2019; 68 (6): 847-853. doi: 10.1097/MPG.0000000000002265
9. Clarkston K, Tsai Y T, Jackson K, Rosen M J, Denson L A, Minar P. Development of Infliximab Target Concentrations during Induction in Pediatric Crohn's Disease Patients. Journal of pediatric gastroenterology and nutrition. Feb. 12 2019; doi: 10.1097/MPG.0000000000002304
10. Xiong Y, Mizuno T, Colman R, et al. Real-World Infliximab Pharmacokinetic Study Informs an Electronic Health Record-Embedded Dashboard to Guide Precision Dosing in Children with Crohn's Disease. Clinical pharmacology and therapeutics. 06 2021; 109 (6): 1639-1647. doi: 10.1002/cpt.2148
11. Colman R J, Samuels A, Mizuno T, Punt N, Vinks A A, Minar P. Model-informed Precision Dosing for Biologics Is Now Available at the Bedside for Patients With Inflammatory Bowel Disease. Inflammatory bowel diseases. Nov. 22 2022; doi: 10.1093/ibd/izac237
12. Cook S F, Bies R R. Disease Progression Modeling: Key Concepts and Recent Developments. Curr Pharmacol Rep. October 2016; 2 (5): 221-230. doi: 10.1007/s40495-016-0066-x
13. Colman R J, Xiong Y, Mizuno T, et al. Antibodies-to-infliximab accelerate clearance while dose intensification reverses immunogenicity and recaptures clinical response in paediatric Crohn's disease. Aliment Pharmacol Ther. March 2022; 55 (5): 593-603. doi: 10.1111/apt.16733
14. Levine A, Griffiths A, Markowitz J, et al. Pediatric modification of the Montreal classification for inflammatory bowel disease: the Paris classification. Inflammatory bowel diseases. June 2011; 17 (6): 1314-21. doi: 10.1002/ibd.21493
15. Mould D R, D′Haens G, Upton R N. Clinical Decision Support Tools: The Evolution of a Revolution. Clinical pharmacology and therapeutics. April 2016; 99 (4): 405-18. doi: 10.1002/cpt.334
16. Neely M, Jelliffe R. Practical, individualized dosing: 21st century therapeutics and the clinical pharmacometrician. J Clin Pharmacol. July 2010; 50 (7): 842-7. doi: 10.1177/0091270009356572
17. Dubinsky M C, Mendiolaza M L, Phan B L, Moran H R, Tse S S, Mould D R. Dashboard-Driven Accelerated Infliximab Induction Dosing Increases Infliximab Durability and Reduces Immunogenicity. Inflammatory bowel diseases. Sep. 1 2022; 28 (9): 1375-1385. doi: 10.1093/ibd/izab285
18. Fasanmade A A, Adedokun O J, Blank M, Zhou H, Davis H M. Pharmacokinetic properties of infliximab in children and adults with Crohn's disease: a retrospective analysis of data from 2 phase III clinical trials. Clin Ther. July 2011; 33 (7): 946-64. doi: 10.1016/j.clinthera.2011.06.002
19. Vande Casteele N, Gils A, Singh S, et al. Antibody response to infliximab and its impact on pharmacokinetics can be transient. The American journal of gastroenterology. June 2013; 108 (6): 962-71. doi: 10.1038/ajg.2013.12
20. Whaley K. Anti-TNF Pharmacokinetics and Response to Therapy in Pediatric Acute Severe Uclerative Colitis (The ARCH Study). In: Xiong V, Karns R, Hyams J, et al, editors. Gastroenterology2021. p. S75-S76.
21. Brandse J F, van den Brink G R, Wildenberg M E, et al. Loss of Infliximab Into Feces Is Associated With Lack of Response to Therapy in Patients With Severe Ulcerative Colitis. Gastroenterology. August 2015; 149 (2): 350-5 e2. doi: 10.1053/j.gastro.2015.04.016
22. Rosen M J, Minar P, Vinks A A. Review article: applying pharmacokinetics to optimise dosing of anti-TNF biologics in acute severe ulcerative colitis. Alimentary pharmacology & therapeutics. June 2015; 41 (11): 1094-103. doi: 10.1111/apt.13175
23. Bartel C L K, Samuels A, Hyams J, Boyle B, Kurowski J, Saeed S, Noe, JD M P. Evaluation of Blood and Stool Biomarkers of

Deep Remission in Pediatric Crohn's Disease Identifies Anti-TNF Clearance as a

Vital Biomarker of Treatment Response. Journal of Pediatric Gastroenterology; 2022.
24. Hyams J, Crandall W, Kugathasan S, et al. Induction and maintenance infliximab therapy for the treatment of moderate-to-severe Crohn's disease in children. Multicenter Study

Randomized Controlled Trial. Gastroenterology. March 2007; 132 (3): 863-73; quiz 1165-6. doi: 10.1053/j.gastro.2006.12.003
25. Fernandes S R, Bernardo S, Simoes C, et al. Proactive Infliximab Drug Monitoring Is Superior to Conventional Management in Inflammatory Bowel Disease. Inflammatory bowel diseases. Jan. 6 2020; 26 (2): 263-270. doi: 10.1093/ibd/izz131
26. Frymoyer A, Hoekman D R, Piester T L, et al. Application of Population Pharmacokinetic Modeling for Individualized Infliximab Dosing Strategies in Crohn Disease. Journal of pediatric gastroenterology and nutrition. December 2017; 65 (6): 639-645. doi: 10.1097/MPG.0000000000001620

All percentages and ratios are calculated by weight unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method for determining a dose of an anti-tumor necrosis factor α (anti-TNFα) biologic for administering to an individual having an inflammatory condition, comprising
a) determining, in the individual, a level of one or more time-varying covariates selected from weight, albumin, erythrocyte sedimentation rate (ESR), neutrophil CD64 (nCD64) expression, and combinations thereof;
b) predicting a future concentration of biologic using a Emax time-varying covariate model; and
c) determining, based on step (b), a dose of the anti-TNFα biologic.

2. The method of claim 1, the inflammatory condition being an inflammatory bowel disease (IBD).

3. The method of claim 1, the inflammatory condition being an inflammatory bowel disease (IBD) selected from Crohn's Disease (CD), pediatric CD, Ulcerative Colitis (UC), Pediatric UC, Pediatric onset IBD, moderate to severe CD or UC, and combinations thereof.

4. The method of claim 1, the inflammatory condition being selected from one or both of uveitis and rheumatoid arthritis.

5. The method of claim 1, the anti-TNFα biologic being an antibody.

6. The method of claim 1, the anti-TNFα biologic having a half-life of from about 10 to about 20 days.

7. The method of claim 1, the anti-TNFα biologic being selected from infliximab, etanercept, and adalimumab.

8. The method of claim 1, the anti-TNFα biologic being infliximab.

9. The method of claim 1, the dose being a dose administered during an induction period.

10. The method of claim 1, the one or more time-varying covariates being at least two of weight, albumin, erythrocyte sedimentation rate (ESR), neutrophil CD64 (nCD64) expression.

11. The method of claim 1, the one or more time-varying covariates being at least three of weight, albumin, erythrocyte sedimentation rate (ESR), neutrophil CD64 (nCD64) expression.

12. The method of claim 1, the one or more time-varying covariates being each of weight, albumin, erythrocyte sedimentation rate (ESR), neutrophil CD64 (nCD64) expression.

13. The method of claim 1, at least one of step (a), step (b), or step (c) being implemented using a computer.

14. The method of claim 1, the individual being under 18 years of age.

15. The method of claim 1, the individual being an adult.

16. The method of claim 1, the individual being anti-TNFα naïve.

17. A method of treating an individual having an inflammatory condition comprising
a. determining a value for a biomarker selected from weight, serum albumin (ALB), erythrocyte sedimentation rate (ESR), and neutrophil CD64 ratio (nCD64) in the individual;
b. simulating a predicted concentration-time curve for the anti-TNFα biologic based on the biomarker value and a correction factor;
c. administering to the individual, a dose of a tumor necrosis factor-alpha inhibitor a (anti-TNFα) biologic based on the predicted concentration-time curve.

18. The method of claim 17, the biologic having a half-life of from about 10 to about 20 days.

19. The method of claim 17, the biologic being selected from infliximab, etanercept, and adalimumab.

20. The method of claim 17, the inflammatory condition being selected from Crohn's Disease (CD), pediatric CD, Ulcerative Colitis (UC), Pediatric UC, Pediatric onset BBD, moderate to severe CD or UC, uveitis, rheumatoid arthritis, and combinations thereof.
